(19) United States
(12) Patent Application Publication
(10) Pub. No.: US 2005/0267689 A1
(43) Pub. Date: Dec. 1, 2005

(54) Method to automatically identify peak and monoisotopic peaks in mass spectral data for biomolecular applications

(76) Inventor: Maxim Tsypin, Steamboat Springs, CO (US)

Correspondence Address: PATTON BOGGS, L.L.P., 2001 ROSS AVENUE, SUITE 3000, DALLAS, TX, 75201, US

(21) Appl. No.: 10/887,138

(22) Filed: Jul. 7, 2004

Related U.S. Application Data
(60) Provisional application No. 60/485,632, filed on Jul. 7, 2003.
(60) Provisional application No. 60/485,633, filed on Jul. 7, 2003.
(60) Provisional application No. 60/485,476, filed on Jul. 7, 2003.

(51) Int. Cl. G06F 19/00; G01N 33/48; G01N 33/50

(52) U.S. Cl. 702/19; 702/27

(57) ABSTRACT

A method for identifying peaks in mass spectral data includes: eliminating non-constant levels of background noise; detecting all peaks above a user-identified signal-to-noise ration threshold; and compiling a list of all detected peaks.

### CROSS-REFERENCE TO RELATED APPLICATIONS

This application claims the benefit of and priority to U.S. Provisional Application No. 60/485,632, filed Jul. 7, 2003, which is hereby incorporated by reference.

This application also incorporates by reference commonly-owned U.S. Provisional Application Nos. 60/485,633 and 60/485,476, both filed on Jul. 7, 2003.

### BACKGROUND OF THE INVENTION

1. Field of the Invention

The present application relates to a method for automatically identifying peaks and monoisotopic peaks in mass spectral data for biomolecular applications.

2. Description of Related Art

Mass spectroscopy has become an important tool in the identification of proteins and other molecules in biological and pharmacological applications. Complexity of the resulting mass spectroscopic data creates new challenges for analysis of the raw data, which exceed the capabilities of existing software solutions.

Mass spectroscopy is a technique that allows the accurate determination of the masses of molecules present in the sample being analyzed. For samples of biological origin, the mass range of interest is typically from 500 to 10000 Da for enzymatically digested proteins (typically, but not limited to, digested by trypsin; 1 Da is the unit of mass equal to 1/12^{th} of the mass of the atom of Carbon-12), and up to 300 kDa for undigested proteins. Raw mass spectral data sets typically have the structure of a sequence of pairs (m/z, signal), where m/z is the mass-to-charge ratio, and signal is proportional to the number of molecules in the sample, that have this particular mass-to-charge ratio. A typical data set can contain from thousands to millions of such (m/z, signal) pairs (data-points), at regularly spaced m/z values. By plotting the data as signal versus individual species of molecules manifest themselves as peaks (denoted by crosses at FIG. 1), where individual species of molecules manifest themselves as peaks (denoted by crosses at FIG. 1). Thus, extracting the information about what molecules are present in the sample, requires detection of peaks in the mass spectrum. As a single spectrum typically contains from dozens to hundreds of peaks (and sometimes even more), and modem mass spectrometers often produce data at a rate of several spectra per second, automation of peak detection becomes essential.

Current state of the art is that while mass spectrometers become more and more advanced in terms of sensitivity, resolution, mass range, mass accuracy, number of peaks that can be resolved in a single spectrum, and the number of spectra acquired per second, the unsatisfactory performance of the state of the art software systems and algorithms for peak detection in mass spectral data becomes a bottleneck in using these mass spectrometers in biological, clinical and pharmacological applications, especially in high-throughput applications.

Existing algorithms typically cannot adjust, or can only poorly adjust, to the widely varying levels of background and noise in the mass spectra. In practice it routinely happens that one has to process large batches of spectra (such as, but not limited to, dozens to hundreds of spectra obtained when proteins in the sample under study are separated by two-dimensional gel electrophoresis, and then each spot on the gel is analyzed by the MALDI mass spectrometer) that have very different levels of background (i.e., baseline) and noise. To make matters worse, background and noise often change substantially not only from spectrum to spectrum, but also within one spectrum.

Thus, with existing peak detection algorithms and software, if one uses the settings appropriate for “noisy” spectra, i.e., for high background and/or high noise (which settings naturally correspond to lower sensitivity), one misses weak peaks in “clean” (low noise) spectra (or regions of spectral). On the other hand, if one uses the settings appropriate for “clean” spectra, i.e., low background and noise (which settings naturally correspond to higher sensitivity), one detects too many false positive peaks in “noisy” spectra (or regions of spectra), that is, spikes in the data actually due to noise are erroneously detected as peaks.

As a result, with existing peak detection algorithms and software, practitioners in the field routinely need to hand-tune parameters of peak detection algorithms, not only from spectrum to spectrum, but also within different m/z regions of single spectra, to adjust them to varying levels of background and noise. The resulting amount of pain-staking labor is a big obstacle in the automated analysis of samples. Essentially, with existing peak detection algorithms and software, practitioners have to choose between automatic, but low quality, peak detection and manually-asserted high-quality peak detection.

Identifying molecules from mass spectroscopic data is further complicated by the fact that atoms constituting molecules of biological interest (these are, typically, Carbon C, Hydrogen H, Nitrogen N, Oxygen O, Sulfur S; sometimes also other chemical elements) have different isotopes characterized by their natural isotopic abundances (for example, approximately 98.9% of carbon atoms are C-12, whose mass is 12 Da, while remaining approximately 1.1% of carbon atoms are C-13, whose mass is 13 Da). Thus, large molecules of biological interest, such as, but not limited to, proteins, DNA and other biopolymers, as well as their fragments obtained by enzymatic or other means, appear in mass spectra not as single peaks, but as groups of peaks which masses differ by 1 Da, known to those skilled in the art as isotopic clusters. Examples of such clusters are clearly visible in FIG. 1, where one can observe a strong isotopic cluster with leftmost peak at 2033 Da, two medium-strength clusters with leftmost peaks at, respectively, 2040 and 2052 Da, and a weak cluster with leftmost peak at 2062 Da. Leftmost peaks in isotopic clusters correspond to molecules containing only the lowest-mass isotopes of all their atoms: all carbon atoms are C-12, all hydrogen atoms are H-1, all nitrogen atoms are N-14, and so on. These peaks are known to those skilled in the art as monoisotopic peaks. While each chemical species of molecule manifests itself in the mass spectrum as an isotopic cluster, it is characterized by only one monoisotopic peak, thus it became common practice to characterize molecules in the mass range of up to approximately 10 kDa by their monoisotopic masses. For example, it became common practice to use monoisotopic masses in protein identification methods based on comparing mass spectral data to databases of masses of protein fragments.

Therefore, for such applications initial peak detection needs to be supplemented by an algorithm that identifies the monoisotopic peak in isotope clusters, which becomes increasingly difficult for higher masses (higher than 3 or 4 kDa), when monoisotopic peak becomes weaker than other peaks in isotopic cluster, and may (for higher masses and weaker signal) be not detectable at all.

### BRIEF SUMMARY OF THE INVENTION

The disclosed algorithms utilize properties of isotopic clusters (relationships between amplitudes of individual peaks in the cluster, as a function of monoisotopic mass) to enhance the reliability and sensitivity of detection of monoisotopic peak, as well as the accuracy of determination of the corresponding monoisotopic mass.

The disclosed peak detection method includes algorithms that automatically estimate non-constant (m/z-dependent) levels of background and noise, detect all peaks above a user-defined signal-to-noise ratio threshold, and compile a list of all detected peaks. In a second step, applicable if the resolving power of the instrument was high enough to resolve peaks within isotopic clusters, yet another algorithm identified monoisotopic peaks.

The ability of our peak detection method and underlying algorithms to automatically compute accurate and robust m/z-dependent estimates for background and noise, and automatically adapt its sensitivity correspondingly, makes it possible to process large batches of spectra that have widely varying levels of background and noise, using the same setting for signal-to-noise ratio threshold. This solves the above-mentioned automation problem: manual intervention to adjust peak detection parameters for individual spectra is no longer required. Moreover, within individual spectrum sensitivity adapts to the local background and noise level: in the noisy regions only sufficiently strong signals are detected, while in low-noise regions sensitivity increases correspondingly to the local noise level.

The above as well as additional objectives, features, and advantages of the present invention will become apparent in the following detailed written description.

### BRIEF DESCRIPTION OF THE DRAWINGS

FIG. 1 illustrates a plot of raw mass spectral data having monoisotopic peaks.

### DETAILED DESCRIPTION OF THE PREFERRED EMBODIMENT

In the following detailed description of the preferred embodiments, reference is made to the accompanying drawings, which form a part hereof and in which is shown by way of illustration specific preferred embodiments in which the invention may be practiced. These embodiments are described in sufficient detail to enable those skilled in the art to practice the invention, and it is understood that other embodiments may be utilized and that logical software, electrical, mechanical, structural, and chemical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the appended claims.

Peak Detection Algorithm

To make disclosed algorithms easier, understandable and reproducible by those skilled in the art, we first give the outline, describing the major steps and underlying procedures, and then elaborate further on important details.

We use abbreviation FWHM to denote Full Width at Half Maximum. It characterizes width of peaks in the mass spectrum.

Our mathematical notation follows conventions used in the C programming language, which is commonly used by those skilled in the art:

>=denotes “greater or equal”,

* denotes multiplication,

&& denotes logical AND,

sqrt (x)denotes square root of x,

a_raw[I] denotes i-th element of array a_raw,

and so on. We often perform certain operations on all data points, for example, subtracting the background from original raw data, and storing results in array. In such cases, for the sake of brevity, we write

a_subtr[i] =a_raw[i] −a_base[i]

and omit explicitly mentioning “for all I”, which is implicitly assumed.

Outline of Peak Detection Algorithm:

0. Input the spectrum profile dataset in which the peaks are to be detected. This is the sequence of (m/z, signal) pairs, typically, but not necessarily, regularly spaced in m/z.

Input user-defined parameters: signal-to-noise ratio threshold, width of the moving window (number of data points) for background and noise computation, peak width.

1. Use robust asymmetric estimates in the moving window to compute background, noise and background-subtracted signal.

2. Compute convolution of background-subtracted signal with (peak shape with its average subtracted). It will be used in the next step to find candidate peak positions, similar to algorithm described in Reference [1].

3. Compute the sum of background-subtracted signal over the peak core. Use the noise estimate from step 1 to compute the noise estimate for this sum. Go through all points and check peak detection criteria:
(a) this should be the global maximum of convolved signal within the window of halfwidth 0.7*peak_FWHM
(b) convolved signal at this m/z should be positive
(c) sum of background-subtracted signal over the peak core is greater than (user-defined signal-to-noise ratio threshold) * (noise estimate for sum of background-subtracted signal over the peak core).

If all three criteria are satisfied, peak is detected:

Add this data point to the list of detected peaks.

4. Refine m/z values for peak positions. Before this step, these values were selected from m/z values of original data points. Now we obtain more accurate m/z values for peak positions that are typically in between m/z values of original data points.

5. Output resulting peak list. For each detected peak, output its position, amplitude, signal-to-noise ratio and possibly other characteristics.

Now we present a more detailed description of the algorithm. For the sake of clarity, this description uses certain concrete numerical values of internal parameters that we used in reduction to practice, such as, but not limited to, 25^{th} and 50^{th} percentiles used for estimating background and noise. It should be understood that disclosed algorithms are general, and not specific to these specific numerical values. For example, one could use 15^{th} and 50^{th} percentile instead of 25^{th} and 50^{th}, with corresponding change to the number 0.6745.

0. Input the spectrum profile dataset in which the peaks are to be detected. This is the sequence of (m/z, signal) pairs, typically, but not necessarily, regularly spaced in m/z.
Store m/z values in array m_z.
Store signal (raw data) values in array a_raw.
Input user-defined parameters:
SNRthreshold—signal-to-noise ratio threshold.
win_width—width of the moving window (number of data points) for background and noise computation.
Peak_FWHM—peak full width at half maximum.

1. Use robust asymmetric estimates in the moving window to compute background, noise and background-subtracted signal.

1.1 For every m/z value collect a set of values of a_raw for all data points in the window centered around this m/z value, compute 25^{th} percentile of this set of values, store result in array a_base. This is the first component of the background. For efficiency reasons, instead of performing this operation at every m/z value, one can do it more sparsely (such as for every n-th point, with suitably chosen n), and then interpolate.
Subtract the first part of background from the raw data, store result in array a_subtr:
a_subtr[i]=a_raw[i]−a_base[i].

1.2 Apply the same procedure as in 1.1 to a_subtr:
compute 25^{th} percentile of a_subtr in the moving window, store results in array a_base2. This is the second component of background.

1.3 Compute 50^{th} percentile (median) of a_subtr in the moving window, store results in array a_med2.

1.4 Compute asymmetric robust estimate for m/z dependent noise level, store result in array a_sigma:
a_sigma[i]−(a_med2[i]−a_base2[i])/0.6745
if (a_sigma[i]==0) a_sigma[i]=1.

1.5 Add up two components of background, computed in 1.1 and 1.2, to get full baseline. Store result in a_base:
a_base[i]=a_base[i]+a_base2[i].

1.6 Subtract full baseline from raw data:
a_subtr[i]=a_raw[i]−a_base[i].

2. Compute convolution of a_subtr with peak shape shifted down by its average, as in Ref. [1], store result in array a_conv.

3.1 Compute sum of a_subtr in the window containing n_peak_core points. We used n_peak_core=peak_FWHM, but this should not restrict the generality of the algorithm.
a_peak_core_sum[i]=
a_subtr[i−n_peak_core/2]+ . . . +a_subtr[i+n_peak_core/2].

3.2 Go through all points and check peak detection criteria. Inter-peak distance is required to be greater than 0.7*peak_FWHM: otherwise two Gaussians cannot be resolved anyway.

i = 0

| | |
|---|---|
| while (i < number of points in the data) | { |

| | |
|---|---|
| if(a_conv[i] > 0 | AND |

a_conv[i] >= each of

a_conv[i−0.7*peak_FWHM] ...
a_conv(i+0.7*peak_FWHM] AND

a_peak_core_sum[i] >=

| | |
|---|---|
| SNRthreshold * a_sigma[i] * sqrt (n_peak_core) | ) |

then

add i to peak list
i = i + 0.7*peak_FWHM

else

i = i + 1

endif
}

4. Refine m/z values for peak positions. For each peak at location i (i is the index of data point), take 3 points: i−1, i, i+1 with corresponding values of m_z and a_conv. Perform quadratic interpolation of a_conv as a function of m_z, and find position of the maximum of interpolated function. Store it as the refined value of m/z for the peak.

5. output 3-column peak list: for all detected peaks,
position=refined m_z.
amplitude=a_peak_core_sum.
Signal-to-noise-ratio=a_peak_core_sum/(a_sigma* sqrt(n_peak_core) )

While the disclosed algorithms use previously known general principles, such as computation and subtraction of background, estimation of noise, and use of signal-to-noise ratio to distinguish signal from noise (see Ref. [1-5]), disclosed algorithms incorporate several crucial improvements over the prior art [1-5] that result in a great improvement in performance.

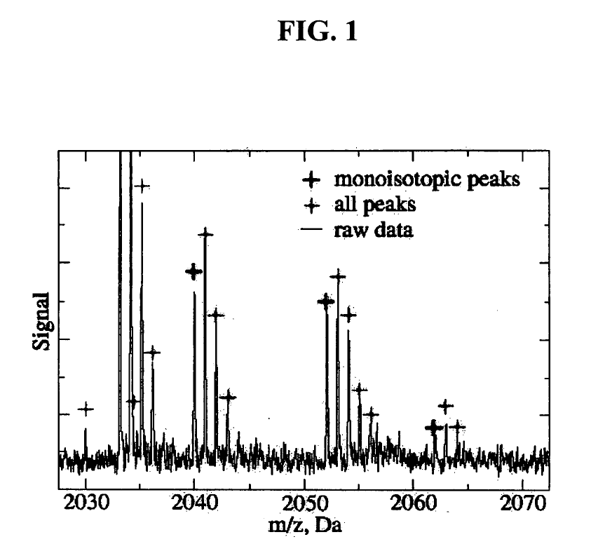

We use robust asymmetric estimates to compute background. Robust means the estimate is based on robust statistics and thus robust against the presence of outlier points. This is especially relevant in processing of mass spectral data, when peaks can be as much as 3 orders of magnitude stronger than surrounding data values, and thus collection of values in the moving window typically contains outliers corresponding to peak values. Also mass spectral data is intrinsically asymmetric, namely, all peaks point up, and there are no peaks pointing down (FIG. 1). In other words, the data typically contains large positive values, which correspond to peaks, but no large negative values. Robust symmetric estimates, such as median in a moving window to estimate background [3], do not take this intrinsic asymmetry of the data into account.

Existing algorithms use estimates that are either non-robust or robust symmetric. References [1], [2] and [4] use the maximum of probability distribution (i.e., mode) to estimate background, which results in non-robust estimate; Ref. [5] uses linear estimates, which are not robust. Ref. [3] uses the estimate (median in the moving window) which is robust, but symmetric. We use robust asymmetric estimate (25^{th} percentile). The resulting advantage is that while symmetric estimate (median) fails when density of peaks becomes high enough so that peaks occupy more than half of data points, our asymmetric estimate works okay up to higher density, namely up to peaks occupying 75% of data points. If necessary, this aspect of performance can be further improved by using percentile lower than 25% (the tradeoff is the decreasing statistical accuracy of the estimate).

We then estimate the background as a sum of two components (see description of algorithm above). While the first component by itself is sufficient for slowly-varying background (namely, background sufficiently flat so that systematic change in the background within the size of moving window is small relative to noise), addition of the second component greatly improves performance for background with significant slope.

We subtract the background from the signal before applying robust asymmetric estimates for noise. This significantly improves the noise estimate when the background has significant slope. Otherwise, due to the slope, the spread of signal values in the moving window becomes larger than just the spread due to noise, and the noise estimate becomes higher than the actual noise.

Our algorithm does not involve iterative refinement or fitting. Thus, it does not have convergence problems, and also works much faster than iterative algorithms, such as Ref. [4].

2.1 Generalization to m/z-dependent peak width.

The above exposition of peak detection algorithm assumes, for the sake of clarity, that user-defined peak width is constant through the spectrum. It is straightforward, however, to generalize the algorithm so that the peak width changes through the spectrum. In this case the user supplies two values for the peak width, corresponding to the beginning (low m/z) and to the end (high m/z) of the spectrum. The program interpolates between these values to compute m/z-dependent peak shape. All other elements of algorithm stay in place. In our reduction to practice we have successfully used this generalized version of the algorithm to process mass spectral data in which peak width did significantly depend on m/z.

2.2 Consistent treatment of non-integer peak width.

When computing quantities such as convolution of background-subtracted signal width (peak shape with its average subtracted) or the sum of background-subtracted signal over the peak core (steps 2 and 3 of peak detection algorithm) it becomes important to treat consistently non-integer peak width, and, in general, the situation when the moving window has non-integer width. This is especially important when the peak width is m/z-dependent, because otherwise we will run into situation that at certain values of m/z the number of data points involved in convolution or in the sum over peak core jumps from one integer value to the neighboring integer. Such discontinuous behavior can easily degrade performance of the peak finder, especially when the jump occurs within an isotopic cluster. To assure continuous behavior, we treat non-integer window size by assigning weights (from 0 to 1) to the leftmost and to the rightmost data points in the window. When the window size grows, these weights grow accordingly. When the weight reaches 1, a new point is included (with weight 0). When the window grows still further, the weight of former rightmost or leftmost data points stays at 1 (now it became a regular internal point), while weight of new point grows.

2.3 Polynomial model for the peak shape.

Convolution of data with the peak shape at step 2 of peak detection algorithm requires the discretized representation of the peak shape—as a set of values at the discrete set of m/z values, corresponding to m/z values of actual data points. As signal values in data points arise essentially from binning of m/z axis, correct discretized representation of the peak shape involves integrating continuous peak shape over m/z bins.

To make this integration computationally efficient, we use a polynomial model of the peak shape (a symmetric 4-th order polynomial approximating the Gaussian). When computing convolution, we use only the central part of peak shape: part above half-maximum (i.e., the part within the FWHM-wide window). This improves detection of poorly-resolved peaks. When computing sum over the peak core, we also use the same window.

3. Algorithm for identification of monoisotopic peaks.

As mentioned in the Introduction, an important step in the application of mass spectroscopy to biomolecular applications is the identification of monoisotopic peaks. As different isotopes differ by 1 Da, the simplest way to identify peaks is to take the first peak in all clusters that exhibit a 1 Da mass spacing, and label the first peak in each cluster as a monoisotopic peak. (Here we consider the data containing only singly-charged ions, as is commonly the case for MALDI spectra; processing of data from multi-charged ions requires further generalization of algorithm).

In the disclosed algorithm we refine this idea using the empirical observation that the amplitude ratios in isotopic clusters of peptides obey certain quantitative relations.

Let's denote by A0 the amplitudes of the monoisotopic peak (located at mass M), and by A1 the amplitude of the next peak (located at mass M+1).

Then, empirically (due to the average atomic composition of peptides) the following relation is fairly accurately satisfied:

A1/A0 =5.56e−4 * M,

where M is the mass of the monoisotopic peak (in Daltons). Thus, the algorithm for identifying monoisotopic peaks is as follows:

Step through all peaks in the peak list and label a peak with mass M and. amplitude Ampl as monoisotopic, if it satisfies both of the following criteria:
1) there is a peak in the window around M+1 Da, no smaller than b * Ampl * (A1/A0).
2) in the window around M-1 Da there is either
   no peak, or
   peak smaller than b * Ampl/(A1/A0).

Here A1/A0=5.56e−4 * M, and algorithm has two empirical parameters: width of the window around M+1 and M−1, which reflects the accuracy of mass determination (reasonable value is 0.2 Da for full width, so that window around M−1 becomes the interval [M+0.9, M+1.1], and window around M−1 becomes the interval [M−1.1, M−0.9]), and parameter b that reflects the accuracy of amplitude determination (reasonable value of b for MALDI spectra is approximately 0.6 to 0.7).

4. Suppression of chemical noise.

In mass spectra, it is often the case that chemical contaminants, fragments due to unspecific cleavage, etc., give rise to many unwanted peaks. In MALDI spectra, this problem often manifests itself as a “peak at (almost) every Dalton” phenomenon. This problem is particularly severe at low m/z (below 1000 Da), where isotopic clusters for weak signals are essentially represented by a single (monoisotopic) peak, higher peaks being too weak to be detected.

In this situation it becomes advantageous to detect only stronger peaks in such overpopulated regions. Thus, in this case we apply additional criteria to the list of monoisotopic peaks:

Step through all monoisotopic peaks and retain the peak with mass M in the final list, if it satisfies either of two criteria:
1) Signal-to-noise ratio>C * SNRthreshold, OR
2) Ratio of peak amplitude to the amplitude of the preceding peak (in the window around M−1, not necessarily monoisotopic) is greater than C.
   Here C is the user-defined parameter (chemical noise suppression factor). The higher the value of C, the stronger suppression of chemical noise.

5. Coherent detection of resolved isotopic clusters at high mass.

At masses higher than approximately 2000 Da the second isotopic peak becomes stronger than monoisotopic. At still higher mass (more than about 4 kDa) it often happens that while isotopic components of isotopic cluster are still resolved, monoisotopic peak can no longer be detected, especially for weak signals.

In this situation, we change our peak detection algorithm as follows. Instead of Gaussian-like profile of individual peak that we used for convolution, we now use the profile of the whole cluster, estimated from the average atomic composition of peptides (“averaging”, see Ref. [6]). Sum over peak core becomes the sum over the cores of the peaks composing the cluster. This makes it possible to detect whole clusters and thus accurately determine position of the monoisotopic peak, even when monoisotopic peak by itself is no longer visible.

6. Summary

The above algorithms allow for an automated peak detection and monoisotopic peak identification in an automated fashion. After a couple of parameters are set the user can run these algorithms without any additional interaction over a large number of spectra. This avoids painstaking manual intervention. The algorithms for monoisotopic peak selection make use of specific numerical parameters reflecting the chemical composition of peptides and natural abundance of isotopes. Thus, to apply the algorithms to non-peptide compounds, as well as to peptides with non-standard isotope composition, one needs to change these parameters accordingly. With this straightforward modification, disclosed algorithms are applicable to a wide range of chemical compounds.

As will be recognized by those skilled in the art, the innovative concepts described in the present application can be modified and varied over a tremendous range of applications, and accordingly the scope of patented subject matter is not limited by any of the specific exemplary teachings given.

While the invention has been particularly shown and described with reference to a preferred embodiment, it will be understood by those skilled in the art that various changes in form and detail may be made therein without departing from the spirit and scope of the invention.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC §112 unless the exact words “means for” are followed by a participle.

1. A method for identifying peaks in mass spectral data comprising the steps of:
estimating non-constant levels of background and noise;
detecting all peaks above a user-defined signal-to-noise ratio threshold; and
compiling a list of all detected peaks.

2. A method for automatically identifying a peak in a mass spectral data set, the method comprising the steps of:
inputting the mass spectral data set;
inputting user-defined parameters including a signal-to-noise ratio threshold, a moving-window width of a moving window through which a portion of the data set is viewed, and a peak width;
compute a background-subtracted signal within the moving window;
compute convolution of the background-subtracted signal;
compute a sum of the background-subtracted signal over a peak core;
for each data point in mass spectral set, compare data point to peak detection criteria;
if the data point satisfies all peak detection criteria, store data point as a detected peak;
refine m/z value for each detected peak; and
output list of detected peaks.
